(19) United States
(12) Patent Application Publication
(10) Pub. No.: US 2010/0209376 A1
(43) Pub. Date: Aug. 19, 2010

(54) Matt Wax

(76) Inventors: Bernd Richters, Hamburg (DE); Dirk Hentrich, Hamburg (DE)

Correspondence Address: Henkel Corporation, 10 Finderne Avenue, Bridgewater, NJ, 08807, US

(21) Appl. No.: 12/768,792

(22) Filed: Apr. 28, 2010

Related U.S. Application Data
(63) Continuation of application No. PCT/EP08/62456, filed on Sep. 18, 2008.

(30) Foreign Application Priority Data
Oct. 31, 2007 (DE) 10 2007 052 391.4

(51) Int. Cl. A61K 8/25; A61K 8/19; A61K 8/97; A61Q 5/00; A61K 8/89

(52) U.S. Cl. 424/70.12; 424/70.1; 424/74

(57) ABSTRACT

Cosmetic agent for temporarily shaping keratin fibers, which contains, in a cosmetically acceptable carrier, 10 to 85% by weight, based on the entire agent, of at least one wax having a melting point in the range of 40° C. to 90° C. and 1 to 15% by weight of at least one composite powder, which is made up of (A) powder particles of a cured organosiloxane composition having an average particle diameter of 0.1 to 200 μm and (B) amorphous silicon dioxide particles having an average particle diameter of not more than 1 μm, the amorphous silicon dioxide (B) particles being immobilized on the surface of the powder particles of the cured organosiloxane composition (A). The invention also relates to the use of said agent for temporarily shaping hair.

### CROSS REFERENCE TO RELATED APPLICATIONS

The present application is a continuation of International Patent Application No. PCT/EP2008/062456, filed 18 Sep. 2008, which claims priority to German Patent Application No. 10 2007 052 391.4, filed 31 Oct. 2007.

The present invention relates to wax-based cosmetic agents for temporary shaping of keratin fibers, in particular human hair, and use of these agents for temporary shaping of hair.

Keratin fibers generally include all animal hair, for example, wool, horsehair, angora hair, fur, feathers and products or textiles manufactured from the same. The term preferably refers to human hair, however.

Hair fixing agents for shaping keratin fibers have long been known and are used in various embodiments to construct, refresh and set hairstyles which, for many hair types, can only be achieved by use of active fixing agents. Such agents are also known as styling agents, with hair treatment agents used for permanent shaping, as well as those used for temporary shaping of hair playing an important role. Temporary shaping effects, which offer good hold without adversely affecting the healthy appearance of the hair, can be achieved, for example, with hair sprays, hair waxes, hair gels, blow-drying, etc.

Hair sprays commonly contain synthetic polymers as the shaping component. These preparations contain a dissolved or dispersed polymer that is applied to the hair by propellants or via a pump mechanism. A satisfactorily uniform distribution of the polymer on the hair is generally achieved in this way; a simple and clean application is possible from spray or pump containers.

Hair waxes generally contain vegetable, animal or mineral waxes as the shaping component and are generally sold as solid formulations, usually in jar containers. To apply these products they are first rubbed in the hand and then spread over the hair. More recently, wax-based preparations have also been developed which can be sprayed. This allows hair waxes to be applied directly to the fibers to be treated. Hair waxes provide good hold for hair due to their natural raw materials, with conventional hair waxes generally imparting a high level of gloss to the hair.

Some consumers, however, do not desire a glossy effect from the use of styling products and, instead, prefer a matt effect. This can be achieved, for example, by providing a high proportion of wax components in the individual styling product. However, such styling products tend to be very sticky. Further, the application of such products with the hands is considered unpleasant, with product residues having to be carefully washed off the hands and the resulting stickiness of the treated hair being undesirable.

Accordingly, the present invention is directed towards cosmetic agents for the temporary shaping of keratin fibers that provide very good hold and a matt effect. The agents further have low stickiness.

According to the present invention, this is achieved by combining certain waxes with composite powders based on cured organosiloxane compositions. Surprisingly, the stickiness of the styling agent is significantly reduced by addition of the composite powder, with excellent hold of the treated keratin fibers being achieved at the same time.

The invention is therefore directed towards cosmetic agents for the temporary shaping of keratin fibers, containing in a cosmetically acceptable carrier
a) 10 to 85 wt. %, based on total weight of the agent, of at least one wax having a melting point in a range from 40° C. to 90° C., and
b) 1 to 15 wt. %, based on total weight of the agent, of at least one composite powder, consisting of
   (A) powdered particles of a cured organosiloxane composition having an average particle diameter of 0.1 to 200 μm, and
   (B) amorphous silicon dioxide particles having an average particle diameter of at most 1 μm,
      wherein the amorphous silicon dioxide particles (B) are immobilized on the surface of the powdered particles of the cured organosiloxane composition (A).

Cosmetic agents according to the invention preferably contain the composite powder b) in an amount of 3 to 12 wt. %, preferably in an amount of 5 to 10 wt. %, based on total weight of the agent.

Composite powders b) have been known for some time. Production of cured silicone powders is described, for example, in EP 0 647 672 A1. These composite powders can be used in a whole host of diverse areas. GB 2 423 250 A discloses their use in cosmetics, focusing substantially on their use in anti-wrinkle creams and other facial and personal care agents.

Composite powders that are used according to the invention consist of
(A) powdered particles of a cured organosiloxane composition having an average particle diameter of 0.1 to 200 μm, and
(B) particles of amorphous silicon dioxide having an average particle diameter of at most 1 μm,
   the particles of amorphous silicon dioxide (B) being immobilized on the surface of the powder particles of the cured organosiloxane composition (A).

Average particle diameter according to the invention refers to the weight average, which is determined by known screen analysis.

Composite powders are extremely free flowing, readily dispersible and have a very low tendency to aggregate. They can be incorporated into cosmetic agents without special equipment or particular process steps. As a general rule, it is sufficient to mix the composite powders with other constituents.

Composite powders wherein the powder particles of the cured organosiloxane composition (A) have an average particle diameter of 0.5 to 100 μm are preferred, particularly 1 to 10 μm.

The organosiloxane composition can be, for example, a gel, elastomer or resin. The organosiloxane composition is preferably a cured silicone elastomer. Powdered particles of the cured organosiloxane composition (A) can come in various shapes, with spherical powdered particles being preferred.

The powdered cured organosiloxane composition (A) can be produced, for example, according to the process taught by EP 0 647 672 A1. Production preferably occurs by reacting an organopolysiloxane (I) having at least two alkenyl groups per molecule with an organopolysiloxane (II) having at least two hydrogen atoms bonded to a silicon atom per molecule.

A compound according to formula (I) is preferably used as the organopolysiloxane (I) having at least two alkenyl groups per molecule—

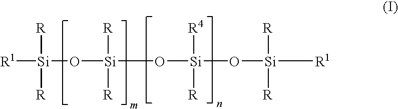

wherein

R is a C₁-C₆ alkyl group or a C₆-C₁₀ aryl group,

R¹ is R or an alkenyl group,

R² is R or an alkenyl group, and

m and n are each independently a whole number from 0 to 5000,

with the proviso that if R¹ is not an alkyl group, R² is an alkyl group and n is at least 2.

A compound according to formula (II) is preferably used as the organopolysiloxane (II) having at least two hydrogen atoms bonded to a silicon atom per molecule

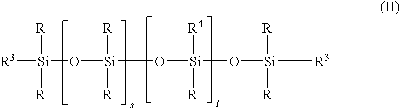

wherein

R has the meaning given above for formula (I),

R³ is R or hydrogen,

R⁴ is R or hydrogen, and

s and t are each independently a whole number from 0 to 5000,

with the proviso that if R³ is not hydrogen, R⁴ is hydrogen and t is at least 2.

Methyl, ethyl, propyl and isopropyl are examples of the C₁-C₆ alkyl group, with methyl being preferred. Suitable C₆-C₁₀ aryl groups include phenyl and naphthyl, with phenyl being preferred. R is preferably methyl in the above formulae.

C₂-C₁₀ alkenyl, such as vinyl, allyl, propenyl, butenyl, pentenyl, hexenyl and decenyl, are examples of the alkenyl group. The alkenyl group is preferably vinyl.

m, n, s and t preferably are each independently a whole number from 0 to 1000, more preferably from 0 to 200.

The powdered cured organosiloxane composition (A) is preferably a dimethicone/vinyl dimethicone copolymer.

Amorphous silicon dioxide particles (B) preferably have an average particle size of 0.001 to 0.1 μm.

Amorphous silicon dioxide particles (B) can be produced by known methods, for example in an electric arc, by a wet-chemical method or by flame hydrolysis. Amorphous silicon dioxide particles (B) are preferably pyrogenic silica (fumed silica).

In order to immobilize amorphous silicon dioxide particles (B) on the powdered cured organosiloxane composition (A), the organosiloxane composition (A) can be converted into an aqueous dispersion, for example, to which the amorphous silicon dioxide (B) is added. Their proportions can be chosen freely within broad limits, with it being preferable to add 0.1 to 30 parts by weight of amorphous silicon dioxide (B) to 100 parts by weight of powdered cured organosiloxane composition (A). The dispersion is heated, preferably to a temperature range of 40 to 95° C. Finally, water is removed to produce the composite powder b).

The commercial product Dow Corning 9701 Cosmetic Powder available from Dow Corning, with the INCI name Dimethicone/Vinyl Dimethicone Crosspolymer and Silica, is particularly preferably used as the composite powder b).

Another component of cosmetic agents according to the present invention is at least one wax having a melting point in a range from 40° C. to 90° C. Generally, all waxes which melt in this temperature range, which satisfy the general definition for waxes as set out, for example, in Ullmanns Encyclopedia of Industrial Chemistry, 4^{th} Ed., Vol. 24, p. 3, left-hand column, and which are physiologically compatible can be used.

The waxes are preferably chosen from vegetable, animal and mineral waxes, with waxes having a melting point in the range from 50° C. to 85° C. being preferred, in particular from 50° C. to 75° C.

Common wax types can be found in known chemical encyclopedias such as the aforementioned Ullmanns encyclopedia.

Particularly preferred waxes according to the invention include beeswax (cera alba), carnauba wax, candelilla wax, montan wax, microcrystalline waxes (microcrystalline paraffins) and cetyl palmitate.

The present invention also includes the combined use of several waxes. Thus, addition of small amounts of carnauba wax can be used to increase the melting and dropping point of another wax and reduce its stickiness. A range of wax blends, optionally mixed with further additives, are commercially available. Examples of such blends preferably used according to the invention are those available under the names “Special Wax 7686 oE” (a blend of cetyl palmitate, beeswax, microcrystalline wax and polyethylene with a melting range from 73 to 75° C.; available from Kahl & Co, Trittau, Germany), Polywax® GP 200 (a blend of stearyl alcohol and polyethylene glycol stearate with a melting point of 47 to 51° C.; available from Croda, East Yorkshire, England) and “Weichceresin® FL 400” (a vaseline/vaseline oil/wax blend with a melting point of 50 to 54° C.; available from Parafluid Mineralolgesellschaft, Hamburg, Germany).

In a particular embodiment of the invention, “liquid waxes” such as jojoba oil can also be used in addition to those compounds conventionally defined as waxes, provided that the melting point of this “wax blend” is not below 40° C.

Cosmetic agents preferably contain waxes in an amount of 10 to 60 wt. %, based on total weight of the agent. Quantities of 10 to 55 wt. % are particularly preferred.

Agents according to the invention contain wax a) and composite powder b) in a cosmetically acceptable carrier.

Preferred cosmetically acceptable carriers include aqueous, alcoholic, or aqueous/alcoholic media. Low alkanols and polyols such as propylene glycol and glycerol can be used as the alcoholic component. Preferred alcohols include ethanol and isopropanol. Water and alcohol can be present in the aqueous/alcoholic base in a weight ratio of 1:10 to 10:1. Water and aqueous/alcoholic mixtures containing up to 50 wt. %, in particular, up to 25 wt. % of alcohol, relative to the alcohol/water mixture, can be preferred bases according to the invention.

Organic solvents or mixture of solvents with a boiling point below 400° C. can be included as additional co-solvents in a quantity of 0.1 to 15 percent by weight, preferably 1 to 10 percent by weight, based on total weight of the agent. Unbranched or branched hydrocarbons such as pentane, hexane and isopentane and cyclic hydrocarbons such as cyclopentane and cyclohexane are particularly suitable as additional co-solvents. Other particularly preferred solvents include glycerol, ethylene glycol, propylene glycol and phenoxyethanol in a quantity of up to 30 wt. % based on total weight of the agent.

Agents according to the invention can consist solely of the cited required constituents. In particular, they can contain the wax as the sole styling component, and in a preferred embodiment they are free of polymers, in particular polymers having a styling effect (i.e., film-forming and/or fixing polymers). Their use is not prohibited, however.

Agents according to the invention can also contain at least one emulsifier. This can be needed if the agent is packed in a relatively liquid, sprayable form. Anionic, ampholytic, cationic and non-ionic surfactants suitable for use on the human body can be used as emulsifiers. Ampholytic surface-active compounds includes zwitterionic surfactants and ampholytes. The use of anionic and non-ionic surface-active compounds is preferred according to the invention.

Anionic surfactants have a water-solubilizing anionic group such as a carboxylate, sulfate, sulfonate or phosphate group and a lipophilic alkyl group having approximately 10 to 22 carbon atoms. The molecule can additionally contain glycol or polyglycol ether groups, ester, ether and amide groups and hydroxyl groups.

Examples of suitable anionic surface-active compounds include, in each case in the form of the sodium, potassium, magnesium and ammonium and also the mono-, di- and trialkanolammonium salts having 2 or 3 carbon atoms in the alkanol group, linear fatty acids (soaps), ether carboxylic acids of the formula R—O—(CH₂—CH₂O)ₙ—CH₂—COOH, wherein R is a linear alkyl group having 10 to 22 carbon atoms and x=0 or 1 to 16, amide ether carboxylates of the formula [R—NH(—CH₂—CH₂—O)ₙ—CH₂—COO]ₘZ, wherein R is a linear or branched, saturated or unsaturated acyl residue having 2 to 29 carbon atoms, n is a whole number from 1 to 10, m is 1 or 2, and Z is a cation from the group of alkali or alkaline-earth metals, acyl sarcosides, acyl taurides, acyl isethionates, sulfosuccinic acid mono- and dialkyl esters, sulfosuccinic acid monoalkyl polyoxyethyl esters, linear alkanesulfonates, linear alpha-olefin sulfonates, alpha-sulfo fatty acid methyl esters, alkyl sulfates and alkyl polyglycol ether sulfates of the formula R—O(—CH₂—CH₂O)ₓ—SO₃H, wherein R is preferably a linear alkyl group having 10 to 18 carbon atoms and x=0 or 1 to 12, mixtures of surface-active hydroxy sulfonates, sulfated hydroxyalkyl polyethylene and/or hydroxyalkylene propylene glycol ethers, sulfonates of unsaturated fatty acids, esters of tartaric acid and citric acid with alcohols, the addition products of approximately 2 to 15 molecules of ethylene oxide and/or propylene oxide with fatty alcohols having 8 to 22 carbon atoms, cocomonoglyceride sulfates, phosphoric acid mono-, di- and triesters of alkoxylated fatty alcohols and mixtures thereof, such as the products sold for example under the Hostaphat® trademark, and esters of hydroxy-substituted bicarboxylic or tricarboxylic acids with polyhydroxylated organic compounds which are selected from the group comprising etherified (C₆-C₁₈) alkyl polysaccharides having 1 to 6 monomeric saccharide units and etherified aliphatic (C₆-C₁₆) hydroxyalkyl polyols having 2 to 16 hydroxyl residues and which are disclosed in European patent EP 0 258 814 B1.

Preferred anionic surfactants include the salts of ether carboxylic acids and phosphate-group-containing compounds, in particular, phosphoric acid mono-, di- and triesters of ethoxylated C₁₀-C₁₈, more particularly, C₁₂-C₁₄, fatty alcohols having degrees of ethoxylation of 2 to 10, in particular, 3 to 5.

Non-ionic surfactants contain as a hydrophilic group a polyol, a polyalkylene glycol ether or a combination of polyol and polyglycol ether groups, for example. Such compounds include—
Addition products of 2 to 30 mol of ethylene oxide and/or 0 to 5 mol of propylene oxide with linear fatty alcohols having 8 to 22 C atoms, fatty acids having 12 to 22 C atoms and alkyl phenols having 8 to 15 C atoms in the alkyl group,
C₁₂-C₂₂ fatty acid monoesters and diesters of addition products of 1 to 30 mol of ethylene oxide with glycerol,
C₈-C₂₂ alkyl monoglycosides and oligoglycosides and ethoxylated analogs thereof and addition products of 5 to 60 mol of ethylene oxide with castor oil and hydrogenated castor oil.

Preferred non-ionic surfactants include addition products of alkylene oxide, in particular, ethylene oxide, with fatty alcohols and fatty acids.

Substances classed as zwitterionic surfactants include those having at least one quaternary ammonium group and at least one —COO⁽⁻⁾ or —SO₃⁽⁻⁾ group in the molecule. Particularly suitable zwitterionic surfactants include betaines such as N-alkyl-N,N-dimethylammonium glycinates, for example, cocoalkyl dimethylammonium glycinate, N-acyl aminopropyl-N,N-dimethylammonium glycinates, and cocoacylaminopropyl dimethylammonium glycinate, and 2-alkyl-3-carboxylmethyl-3-hydroxyethyl imidazolines each having 8 to 18 carbon atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

Ampholytes are substances which, in addition to a C₈-C₁₈ alkyl or acyl group in the molecule, contain at least one free amino group and at least one —COOH or —SCO₃H group and are capable of forming internal salts. Examples of suitable ampholytes include N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 18 carbon atoms in the alkyl group. Preferred ampholytes include N-cocoalkyl aminopropionate, cocoacyl aminoethyl aminopropionate and C₁₂₋₁₈ acyl sarcosine.

Examples of cationic surfactants include in particular quaternary ammonium compounds. Ammonium halides are preferred, in particular chlorides and bromides such as alkyl trimethylammonium chlorides, dialkyl dimethylammonium chlorides and trialkyl methylammonium chlorides, for example, cetyl trimethylammonium chloride, stearyl trimethylammonium chloride, distearyl dimethylammonium chloride, lauryl dimethylammonium chloride, lauryl dimethyl benzyl ammonium chloride and tricetyl methylammonium chloride. Furthermore, highly biodegradable quaternary ester compounds such as are sold under the Dehyquart® trademark and quaternized protein hydrolysates and silicone compounds can be used according to the invention.

Compounds having alkyl groups which are used as surfactants can each be uniform substances. It is generally preferable, however, to use native vegetable or animal raw materials as starting products for these substances so that mixtures of substances having differing alkyl chain lengths depending on the individual raw material are obtained.

Products having a “normal” homolog distribution as well as those having a concentrated homolog distribution can be used for the surfactants which are addition products of ethylene and/or propylene oxide with fatty alcohols or derivatives of these addition products. “Normal” homolog distribution refers to mixtures of homologs which are obtained by reacting fatty alcohol and alkylene oxide using alkali metals, alkali hydroxides or alkali alcoholates as catalysts. Concentrated homolog distributions are obtained when, for example, hydrotalcites, alkaline-earth metal salts of ether carboxylic acids, alkaline-earth oxides, hydroxides or alcoholates are used as catalysts.

The emulsifiers are preferably used in amounts of 0.1 to 10 wt. %, particularly preferably in amounts of 0.5 to 5 wt. %, and most particularly preferably in quantities of 0.7 to 3 wt. %, based on total weight of the agent.

The agents can also contain at least one oil component selected from vegetable, mineral and synthetic oils. These oil components can be added to influence the consistency of the agent. However, as the oil components generally impart gloss to the treated fibers, the quantity of oil component chosen must not be so high that the desired matt effect is lost. These oil components are therefore preferably present in agents according to this embodiment in amounts of less than 15 wt. %, particularly less than 10 wt. %, based on weight of the total agent.

Natural and synthetic oil components include—
Vegetable oils. Such oils include sunflower oil, olive oil, soya oil, rapeseed oil, almond oil, jojoba oil, orange oil, wheat germ oil, peach-kernel oil and the liquid components of coconut butter. Other triglyceride oils are also suitable, such as the liquid components of beef fat and synthetic triglyceride oils.
Liquid paraffin oils, isoparaffin oils and synthetic hydrocarbons, as well as di-n-alkyl ethers having from 12 to 36 carbon atoms, in particular from 12 to 24 carbon atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether. Commercially available products such as 1,3-di-(2-ethylhexyl)cyclohexane (Cetiol® S) and di-n-octyl ether (Cetiol® OE) can be preferred.
Ester oils. Ester oils refer to the esters of C₆-C₃₀ fatty acids with C₂-C₃₀ fatty alcohols. The monoesters of fatty acids with alcohols having 2 to 24 carbon atoms are preferred. Examples of fatty acid components used in the esters include hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof, which form, for example, in the pressurized cleavage of natural fats and oils, in the oxidation of aldehydes from the Roelen oxo synthesis or the dimerization of unsaturated fatty acids. Examples of fatty alcohol components in the ester oils include isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof which form, for example, in the high-pressure hydrogenation of technical methyl esters based on fats and oils or aldehydes from the Roelen oxo synthesis and as monomer fractions in the dimerization of unsaturated fatty alcohols. Particularly preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
Dicarboxylic acid esters such as di-n-butyl adipate, di-(2-ethylhexyl) adipate, di-(2-ethylhexyl) succinate and diisotridecyl acelate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate.
Symmetrical, asymmetrical or cyclic esters of carbonic acid with fatty alcohols, as described, for example, in DE-OS 197 56 454, glycerol carbonate or dicaprylyl carbonate (Cetiol® CC).
Tri-fatty acid esters of saturated and/or unsaturated linear and/or branched fatty acids with glycerol.
Fatty acid partial glycerides, namely monoglycerides, diglycerides and technical mixtures thereof. If technical products are used, small amounts of triglycerides can also be included for production reasons. Partial glycerides are preferably according to formula (D4-I),

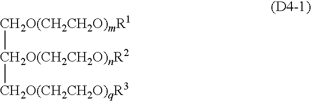

wherein R¹, R² and R³ are each independently hydrogen or a linear or branched, saturated and/or unsaturated acyl residue having 6 to 22, preferably 12 to 18, carbon atoms, with the proviso that at least one of these groups is an acyl residue and at least one of these groups is hydrogen. The sum (m+n+q) is 0 or a number from 1 to 100, preferably 0 or 5 to 25. R¹ preferably is an acyl residue, R² and R³ hydrogen and the sum (m+n+q) is preferably 0. Typical examples include mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Oleic acid monoglycerides are preferably used.

In a preferred embodiment, agents according to the invention contain isopropyl myristate as the oil component.

Depending on desired consistency, agents according to the invention can additionally contain at least one thickening agent.

Suitable thickening agents include agar-agar, guar gum, alginates, xanthan gum, gum arabic, karaya gum, carob seed meal, linseed gums, dextrans, cellulose derivatives such as methyl cellulose, hydroxyalkyl cellulose and carboxymethyl cellulose, starch fractions and derivatives such as amylose, amylopectin and dextrins, clays such as bentonite, fully synthetic hydrocolloids such as polyvinyl alcohol and optionally crosslinked polyacrylates. Particularly suitable thickening agents include hydroxyethyl cellulose and derivatives thereof.

Although it is not preferred, the addition of film-forming and/or fixing polymers is also possible.

All known film-forming and/or fixing polymers can be used. These can be permanently and temporarily cationic, anionic, non-ionic or amphoteric. When at least two film-forming and/or fixing polymers are used, they can naturally have differing charges. For example, it can be preferred to use an ionic film-forming and/or fixing polymer together with an amphoteric and/or non-ionic film-forming and/or fixing polymer. Use of at least two oppositely charged film-forming and/or fixing polymers is also preferred. In the latter case a particular embodiment can in turn additionally contain at least one amphoteric and/or non-ionic film-forming and/or fixing polymer.

As polymers are commonly multifunctional, their functions cannot always be clearly and unambiguously separated from one another. This is particularly true of film-forming and fixing polymers. Nevertheless, a number of film-forming polymers are described by way of example. It is explicitly stated at this point, however, that in the context of the present invention both film-forming and fixing polymers are substantial. As the two properties are also not entirely independent of one another, the term “fixing polymers” also always includes “film-forming polymers” and vice versa.

One of the preferred properties of film-forming polymers is film formation. Film-forming polymers are understood to be polymers which leave behind a continuous film on the skin, hair or nails when they dry. Such film formers can be used in a wide range of cosmetic products, such as for example face masks, make-up, hair fixing agents, hair sprays, hair gels, hair waxes, hair tonics, shampoos or nail varnishes. Such polymers are preferred in particular which have an adequate solubility in alcohol or water/alcohol mixtures in order for them to be present in the agent according to the invention in completely dissolved form. The film-forming polymers can be of synthetic or natural origin.

Film-forming polymers also include polymers which, when used in a 0.01 to 20 wt. % aqueous, alcoholic or aqueous-alcoholic solution, are capable of depositing a transparent polymer film on the hair. These film-forming polymers can be anionically, amphoterically, non-ionically, permanently cationically or temporarily cationically charged.

Suitable synthetic, film-forming, hair-fixing polymers include homopolymers or copolymers constructed from at least one of the following monomers: vinyl pyrrolidone, vinyl caprolactam, vinyl esters such as for example vinyl acetate, vinyl alcohol, acrylamide, methacrylamide, alkyl and dialkyl acrylamide, alkyl and dialkyl methacrylamide, alkyl acrylate, alkyl methacrylate, propylene glycol or ethylene glycol, the alkyl groups of these monomers preferably being C₁ to C₇ alkyl groups, particularly preferably C₁ to C₃ alkyl groups.

Homopolymers of vinyl caprolactam, vinyl pyrrolidone or N-vinyl formamide are cited as examples. Further suitable synthetic film-forming, hair-fixing polymers include copolymers of vinyl pyrrolidone and vinyl acetate, terpolymers of vinyl pyrrolidone, vinyl acetate and vinyl propionate, polyacrylamides, available under the trade names Akypomine® P 191 by CHEM-Y, Emmerich, or Sepigel® 305 by Seppic; polyvinyl alcohols available under the trade names Elvanol® by Du Pont or Vinci® 523/540 by Air Products, and polyethylene glycol/polypropylene glycol copolymers available under the trade names Ucon® by Union Carbide.

Suitable natural film-forming polymers include cellulose derivatives such as hydroxypropyl cellulose having a molecular weight of 30,000 to 50,000 g/mol, available under the trade name Nisso Sl® by Lehmann & Voss, Hamburg, Germany.

Fixing polymers contribute to maintaining and/or establishing volume and fullness in the overall hairstyle. These fixing polymers are also film-forming polymers and are therefore generally typical substances for hair treatment agents used for shaping, such as hair fixing agents, hair mousses, hair waxes, hair sprays. Film formation can be localized and bond only a few fibers together.

Substances which also impart hydrophobic properties to the hair are preferred because as they reduce the tendency of the hair to absorb moisture (i.e., water). This prevents strands of hair from drooping, ensuring lasting shape and hold for the hairstyle. The curl retention test is often used as a test method here. These polymeric substances can also be incorporated into leave-on and rinse-off hair conditioners or shampoos. As polymers are frequently multifunctional (i.e., they demonstrate multiple effects which are desirable from an applications perspective), many polymers are categorized into more than one group classified by mode of action, including in the CTFA handbook. Due to the significance of the fixing polymers, they should therefore be listed according to their INCI names. This list of preferred polymers for use according to the invention also includes the aforementioned film-forming polymers.

Examples of common film-forming, fixing polymers include Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate.

Preferred film-forming and/or fixing polymers include polyvinyl pyrrolidones, vinyl pyrrolidone/vinyl acetate copolymers, vinyl acetate/crotonic acid copolymers, vinyl caprolactam/vinyl pyrrolidone/dimethylaminoethyl methacrylate copolymers, octylacrylamide/acrylates/butylaminoethyl methacrylate copolymers and quaternized vinyl pyrrolidone/dimethyl aminoethyl methacrylate copolymers.

Preferably, the film-forming and/or fixing polymer is the vinyl pyrrolidone/vinyl acetate copolymer Luviskol® VA 37 or PVP/VA copolymer 60/40 W NP, the vinyl acetate/crotonic acid copolymer sold under the trade name Aristoflex® A 60, the vinyl caprolactam/vinyl pyrrolidone/dimethylaminoethyl methacrylate copolymer with the trade name Advantage® LC-E, the amphoteric octylacrylamide/acrylates/butylaminoethyl methacrylate copolymer available under the name Amphomer® or the vinyl pyrrolidone/dimethylaminoethyl methacrylate copolymer quaternized by reaction with diethyl sulfate, which is sold under the trade name Gafquat® 755N.

Agents according to the invention can also contain auxiliary substances, active ingredients and additives conventionally added to customary styling aids.

Suitable auxiliary substances, active ingredients and additives include conditioning agents.

Silicone oil and/or silicone gum can be used as a conditioning agent. However, it is important to choose the quantity and type of component so that the desired matting effect is retained.

Examples of suitable silicone oils or silicone gums include dialkyl and alkylaryl siloxanes such as dimethyl polysiloxane and methyl phenyl polysiloxane, and the alkoxylated, quaternized or also anionic derivatives. Cyclic and linear polydialkyl siloxanes, the alkoxylated and/or aminated derivatives thereof, dihydroxypolydimethyl siloxanes and polyphenyl alkyl siloxanes are preferred.

Silicone oils provide a wide variety of effects. For example, they influence both dry and wet combability of hair, the handle of dry and wet hair, and the gloss. The term “silicone oil” is understood by the person skilled in the art to mean various structures of organosilicon compounds. It includes dimethiconols and dimethicones such as the PEG-12 dimethicone sold by Dow Corning under the name Dow Corning® 193 Surfactant. These can be both linear and branched, and also cyclic or cyclic and branched. It also includes dimethicone copolyols such as those sold by Dow Corning under the name Dow Corning® 5330 Fluid, and aminofunctional silicones, in particular, r the silicones which come under the INCI name Amodimethicone.

Conditioning polymers such as amphoteric polymers are also suitable as conditioning agents.

At least one vitamin, provitamin, vitamin precursor and/or derivative thereof can be used as a conditioning agent.

Preferred vitamins, provitamins and vitamin precursors are conventionally assigned to groups A, B, C, E, F and H. Vitamins belonging to the B group or B complex are particularly preferred, most particularly vitamin B₅ (pantothenic acid, panthenol and pantolactone).

At least one plant extract can be used as a conditioning agent.

These extracts are conventionally produced by extraction of the entire plant. It can also be preferable in individual cases, however, to produce the extracts exclusively from flowers and/or leaves of the plant.

Preferred plant extracts according to the invention include those listed in the table beginning on page 44 of the 3^{rd} edition of the Leitfaden zur Inhaltsstoffdeklaration kosmetischer Mittel, published by the Industrieverband Körperpflege- und Waschmittel e.V. (IKW), Frankfurt.

Extracts from water lily, green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root are preferred.

A range of carboxylic acids are further suitable as a conditioning agent.

Short-chain carboxylic acids can be advantageous. Short-chain carboxylic acids and derivatives thereof include carboxylic acids which can be saturated or unsaturated and/or straight-chain or branched or cyclic and/or aromatic and/or heterocyclic and which have a molecular weight of less than 750. Saturated or unsaturated straight-chain or branched carboxylic acids having a chain length of 1 to 16 carbon atoms in the chain can be preferred within the meaning of the invention, with those having a chain length of 1 to 12 carbon atoms in the chain being most particularly preferred.

Other suitable conditioning agents include protein hydrolysates and/or derivatives thereof, with protein hydrolysates of vegetable origin (e.g., soya, almond, pea, potato and wheat protein hydrolysates) being preferred. Such products are available, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Although use of protein hydrolysates is preferred, other amino acid mixtures obtained by other means can optionally be used in their place. Use of derivatives of protein hydrolysates, for example, in the form of their fatty acid condensation products, is likewise possible. Such products are sold for example under the names Lamepon® (Cognis), Lexein® (Inolex), Crolastin® (Croda), Crosilk® (Croda) or Crotein® (Croda).

The teaching according to the invention naturally encompasses all isomeric forms of hydrolysates, such as cis-trans isomers, diastereomers and chiral isomers.

It is also possible according to the invention to use a mixture of several protein hydrolysates.

In addition to the conditioning agents further auxiliary substances, active ingredients and additives can be added.

By addition of a UV filter, both the agents themselves and the treated fibers can be protected from damaging UV radiation. It can therefore be advantageous to add at least one UV filter to the hair fixing agents. There are no general restrictions on the suitable UV filters in terms of their structure and their physical properties. All UV filters for use in the cosmetics sector whose absorption maximum is in the UVA (315-400 nm), UVB (280-315 nm) or UVC 280 nm) range are suitable. UV filters having an absorption maximum in the UVB range, in particular in the range from approximately 280 to approximately 300 nm, are particularly preferred.

Preferred UV filters according to the invention include substituted benzophenones, p-aminobenzoic acid esters, diphenyl acrylic acid esters, cinnamic acid esters, salicylic acid esters, benzimidazoles and o-aminobenzoic acid esters. Examples include 2-Hydroxy-4-methoxybenzophenone-5-sulfonic acid and the sodium salt thereof (benzophenone-4; Uvinul®MS 40; Uvasorb®S 5).

The addition of perfume components and preservatives is also possible.

The agents can also contain alkalizing agents, conventionally alkali or alkaline-earth hydroxides, ammonia or organic amines. Preferred alkalizing agents are monoethanolamine, monoisopropanolamine, 2-amino-2-methyl propanol, 2-amino-2-methyl-1,3-propanediol, 2-amino-2-ethyl-1,3-propanediol, 2-amino-2-methyl butanol and triethanolamine and also alkali and alkaline-earth hydroxides. Monoethanolamine, triethanolamine and also 2-amino-2-methyl propanol and 2-amino-2-methyl-1,3-propanediol are preferred in particular within the context of this group. The use of ω-amino acids such as ω-aminohexanoic acid as an alkalizing agent is also possible.

Colored pigments or other dyes can also be added to the agents. The pigments having the C.I. names Pigment Red 57:1, Pigment Red 57:2. Pigment Red 172, Pigment Red 90:1, Pigment Yellow 100, Pigment Yellow 115, Pigment Red 174, Pigment Red 4, Pigment Blue 29, Pigment Violet 15, Pigment Violet 16, Pigment Red 29, Pigment Green 17, Pigment Green 18, Natural Red 4, Pigment White 6, Pigment White 14 and Pigment White 31 for example are suitable as colored pigments.

Agents according to the invention can be formulated in forms commonly used for cosmetic agents, for example, in the form of dispersions which can be applied to the hair as a hair lotion or as a pump or aerosol spray, in the form of solid waxes, gels or creams. Formulation as a solid hair wax or paste-like cream is preferred.

If the agent is formulated as an aerosol spray, a propellant should be included as a further component.

The propellant can be advantageously selected so that it also serves as a solvent for the wax components. Such propellants can serve as a solvent for the wax components if they are soluble in the propellant to at least 1 wt. %, relative to the propellant, at 20° C.

Preferred propellants according to the invention include alkanes having 3 to 5 carbon atoms, such as propane, n-butane, isobutane, n-pentane and isopentane; n-butane, propane and mixtures thereof are particularly preferred.

According to a particular embodiment, the agents contain alkanes or mixtures of the alkanes as the sole propellant. However the invention expressly also encompasses the incorporation of propellants of the chlorofluorocarbon type, in particular, fluorocarbons. Dimethyl ethers, preferably in quantities of 5 wt. % or less, based on total weight of the agent, can also be included as an additional propellant.

The propellants are preferably present in amounts of 40 to 98 wt. %, based on total weight of the agent. Quantities of 50 to 95 wt. %, in particular 60 to 90 wt. %, are particularly preferred.

A second subject-matter of the invention is a method for the temporary shaping of hair using the cosmetic agents according to the invention.

The examples below are provided to further illustrate the subject-matter of the present invention without in any way limiting it.

### EXAMPLES

Unless otherwise specified, quantities given below are percentages by weight.

### 1. Wax-Based Styling Pastes—

Styling pastes E1 to E4 were produced in accordance with the Table below:

| Raw materials | E1 | E2 | E3 | E4 |
|---|---|---|---|---|
| Tylose H 100000 YP2 ¹ | 0.10 | 0.10 | 0.10 | 0.10 |
| Phenoxyethanol | 0.60 | — | — | — |
| Euxyl K 300 ² | — | 0.90 | 0.90 | 0.90 |
| AMP-Ultra PC 2000 ³ | 0.15 | 0.15 | — | — |
| AMP-Ultra PC 1000 ⁴ | — | — | 0.15 | 0.15 |
| Colorona Blackstar Gold ⁵ | — | — | 0.10 | 0.10 |
| Cutina MD ⁶ | 5.00 | 9.70 | 5.00 | 5.00 |
| Estol 3752 ⁷ | 1.00 | 2.90 | 1.00 | 1.00 |
| Edenor L2SM ⁸ | 2.00 | 3.90 | 2.00 | 2.00 |
| Isopropylmyristate | 5.60 | 4.90 | 5.60 | 5.60 |
| Weichceresin FL 400 ⁹ | 4.00 | 12.10 | 4.00 | 4.00 |
| Beeswax ¹⁰ | 7.50 | 19.55 | 7.50 | 7.50 |
| Candelilla wax ¹¹ | 3.00 | 6.80 | 3.00 | 3.00 |
| Carnauba wax prima yellow ¹² | 5.00 | 9.70 | 5.00 | 5.00 |
| Dow Corning 9701 Cosmetic | 7.50 | 7.50 | 10.00 | 5.00 |
| Powder ¹³ | | | | |
| FD&C Green No. 3 ¹⁴ | 0.0003 | — | — | — |
| Dekafald ¹⁵ | 0.20 | — | — | — |
| Perfume | 1.50 | 1.80 | 1.80 | 1.80 |
| Water, demineralized | to 100 | to 100 | to 100 | to 100 |

¹ Modified cellulose (90% active substance content; INCI name: Hydroxyethylcellulose) (Shin Etsu)
² Mixture of phenoxyethanol, methyl paraben, butyl paraben, ethyl paraben, propyl paraben and isobutyl paraben (Schülke & Mayr)
³ 2-Amino-2-methyl propanol (INCI name: Aminomethyl Propanol) (Dow Chemical)
⁴ 2-Amino-2-methyl propanol (INCI name: Aminomethyl Propanol) (Dow Chemical)
⁵ Colored pigment consisting of approx. 55-62 wt. % mica and 38-45 wt. % iron oxide (INCI name: Mica and Iron Oxides) (Merck, Rona-EMD)
⁶ Glycerol mono-/distearate (INCI name: Glyceryl Stearate) (Cognis)
⁷ Ethoxylated beeswax (INCI name: PEG-8 Beeswax) (Uniqema)
⁸ Mixture of palmitic and stearic acid (INCI name: Palmitic Acid and Stearic Acid) (Cognis)
⁹ Vaseline/vaseline oil/wax blend (INCI name: Petrolatum) (PARAFLUID MINERALÖLGES.)
¹⁰ INCI name: Beeswax (Kahl & Co.)
¹¹ Wax from Euphorbia cerifera (INCI name: Euphorbia Cerifera (Candelilla) Wax) (Strahl & Pitsch)
¹² Wax from Copernica cerifera (INCI name: Copernica Cerifera (Carnauba) Wax) (Kahl & Co.)
¹³ Composite powder consisting of silicone elastomer particles, to the surface of which amorphous silicon dioxide particles are fixed (INCI name: Dimethicone/Vinyl Dimethicone Crosspolymer and Silica) (Dow Corning)
¹⁴ Triphenylmethane dye (INCI name: Green 3) (Noveon)
¹⁵ 1,3-Dihydroxymethyl-5,5-dimethyl hydantoin (approx. 54-56 wt. % active substance in water; INCI name: DMDM Hydantoin) (Jan Dekker)

2. Wax-Based Styling Paste with Film-Forming Polymer—

Styling paste E5 according to the invention was produced in accordance with the following Table:

| Raw materials | E5 |
|---|---|
| 1,2-Propanediol | 0.20 |
| Propyl paraben | 0.20 |
| Methyl paraben | 0.20 |
| Pemulen TR 1 ¹⁶ | 0.15 |
| Weichceresin FL 400 ⁹ | 6.00 |
| PVP/VA copolymer 60/40 W ¹⁷ | 6.00 |
| Procetyl AWS ¹⁸ | 1.00 |
| Paraffinum Liquidum ¹⁹ | 1.20 |
| Benzoic acid-C₁₂-C₁₅-alkyl ester | 5.00 |
| Edenor L2SM ⁸ | 7.50 |
| Crodafos CES ²⁰ | 1.00 |
| Oleth-20 | 1.00 |
| Beeswax ¹⁰ | 5.00 |
| Lorol C16 ²¹ | 2.00 |
| Brij 72 V ²² | 1.30 |
| Brij 721 VP ²³ | 1.30 |
| AMP-Ultra PC 1000 ⁴ | 0.125 |
| Dekafald ¹⁵ | 0.30 |
| Dow Corning 9701 Cosmetic Powder ¹³ | 5.00 |
| Perfume | 0.90 |
| Water, demineralized | to 100 |

¹⁶ Crosslinked acrylic acid polymer (INCI name: Acrylates/C 10-30 Alkyl Acrylate Crosspolymer) (Lubrizol, Noveon)
¹⁷ Vinyl pyrrolidone/vinyl acetate copolymer (approx. 48-52% solids in water; INCI name: VP/VA Copolymer) (ISP)
¹⁸ Alkoxylated hexadecanol (INCI name: PPG-5-Ceteth-20) (Croda)
¹⁹ Mineral oil (INCI name: Paraffinum Liquidum) (Cognis)
²⁰ INCI name: Cetearyl Alcohol, Dicetyl Phosphate, Ceteth-10 Phosphate (Croda)
²¹ INCI name: Cetyl Alcohol (Cognis)
²² Ethoxylated stearyl alcohol (INCI name: Steareth-4) (Uniqema)
²³ Ethoxylated stearyl alcohol (INCI name: Steareth-21) (Uniqema)

We claim:

1. Cosmetic agent for temporary shaping of keratin fibers, containing in a cosmetically acceptable carrier, relative in each case to the entire agent,
a) 10 to 85 wt. % of at least one wax having a melting point in a range of from 40° C. to 90° C., and
b) 1 to 15 wt. % of at least one composite powder consisting of
(A) powdered particles of a cured organosiloxane composition having an average particle diameter of 0.1 to 200 μm and
(B) amorphous silicon dioxide particles having an average particle diameter of 1 μm or less,
wherein the particles of amorphous silicon dioxide (B) are immobilized on the surface of the powder particles of the cured organosiloxane composition (A).

2. Cosmetic agent according to claim 1, wherein the composite powder b) is present in an amount of 3 to 12 wt. %, based on total weight of the agent.

3. Cosmetic agent according to claim 1, wherein the powdered particles of the cured organosiloxane composition (A) have an average particle diameter of 0.5 to 100 μm.

4. Cosmetic agent according to claim 1, wherein the amorphous silicon dioxide particles (B) have an average particle diameter of 0.001 to 0.1 μm.

5. Cosmetic agent according to claim 1, wherein the cured organosiloxane composition is a cured silicone elastomer.

6. Cosmetic agent according to claim 1, wherein the cured organosiloxane composition is a dimethicone/vinyl dimethicone copolymer.

7. Cosmetic agent according to claim 1, wherein the wax is chosen from vegetable, animal and mineral waxes.

8. Cosmetic agent according to claim 1, wherein the wax is chosen from beeswax, carnauba wax, candelilla wax, montan wax and/or cetyl palmitate.

9. Cosmetic agent according to claim 1, wherein the wax is present in an amount of from 10 to 60 wt. %, based on total weight of the agent.

10. Method of temporary shaping of hair comprising applying the cosmetic agent according to claim 1 to hair.
